(19) United States
(12) Patent Application Publication
(10) Pub. No.: US 2015/0056606 A1
(43) Pub. Date: Feb. 26, 2015

(54) TISSUE TESTER

(71) Applicant: Linda Marie Petter, Auburn, WA (US)

(72) Inventor: Linda Marie Petter, Auburn, WA (US)

(21) Appl. No.: 13/970,932

(22) Filed: Aug. 20, 2013

(51) Int. Cl. C12Q 1/04; B32B 38/08; B32B 38/00; D21H 19/38; B32B 37/16

(52) U.S. Cl. CPC C12Q 1/04; D21H 19/38; B32B 37/16; B32B 38/0004; B32B 38/08; B32B 2260/04; B32B 2554/00
USPC 435/5; 435/34; 435/287.8; 427/2.11; 156/264

(57) ABSTRACT

A tissue tester for providing an indication of the presence of disease including a first paper tissue sheet; and a filter patch treated with a color changing indicator affixed to the first paper tissue sheet. The tester may also consist of a single sheet of filter paper treated with a solution of turmeric and alcohol. The color indicator changes from yellow to brown when disease is indicated in respiratory secretions.

### TECHNICAL FIELD

The present invention relates to a tissue tester providing an early indication of illness, more particularly, the present invention provides an early indication, drug free, self-administered test to determine when an individual may harbor respiratory pathogens that may induce illness.

### BACKGROUND

It would be highly advantageous in the current costly medical/health environment to have access to an inexpensive self-administered home test to determine the need to visit a physician or clinic. It is believed that no such economical and simple drug free test exists. However, the existence of such a self-administered test would be highly advantageous to those experiencing symptoms of illness.

Known testing systems usually require laboratory analysis and expensive equipment. They are not typically available nor amenable to home use. Further, most such tests are limited to detecting specific disease conditions. For example, a patent application entitled “Method for the detection of influenza a/b viruses,” Pub. No. US 20030143530 A1, Pub. date Jul. 31, 2003, having inventors Juergen Klepp et al. concerns a method and a test kit for the detection of an infection by the influenza A and/or influenza B virus containing: i) a device for the collection of a saliva sample and ii) reagents and auxiliary agents for the detection of influenza A and/or influenza B viruses.

The present invention overcomes the deficiencies of the prior art and provides, for the first time, an economical, drug free home test for providing a substantially immediate color indication of an illness. Thus the present invention provides a solution to a long felt need in the health field.

### BRIEF SUMMARY OF THE DISCLOSURE

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, a tester for providing an indication of the presence of disease comprising a filter paper sheet treated in a solution of turmeric and alcohol having a ratio of about 20 ml turmeric to about 237 ml alcohol.

In another aspect the treated filter patch comprises white filter paper.

In another aspect the at least a portion of the tester changes color when exposed to respiratory secretions containing a pathogen.

In another aspect the color change is from a yellow hue to a brown hue.

In another aspect the filter paper sheet is treated by soaking it in the solution for up to 30 seconds.

In one aspect, a tissue tester system is disclosed for providing an indication of the presence of disease. A first paper tissue sheet has a filter patch treated with a color changing indicator affixed to the first paper tissue sheet.

In another aspect, the filter patch is juxtaposed between the first paper tissue sheet and a second paper tissue sheet to form a tissue tester.

In another aspect, the treated filter patch is treated in a solution of turmeric and alcohol.

In another aspect, the treated filter patch is affixed to a sheet of single ply or multi-ply tissue paper by a fixative or method selected from the group consisting of adhesive, glue, paste, tape, hook and loop material, threading, embossing and combinations thereof.

In another aspect, the treated filter patch comprises an area smaller than the first paper tissue sheet.

In another aspect, the treated filter patch is treated in a solution having a ratio of about 20 ml turmeric to about 237 ml alcohol.

In another aspect, the treated filter patch comprises white filter paper.

In yet another aspect, a method for making an early indication, drug free, self-administered tester patch for use in a tester to determine when an individual may harbor respiratory pathogens that may induce illness is disclosed including:

mixing turmeric and alcohol in solution;

soaking filter paper in the solution;

removing the filter paper from the solution; and

drying the filter paper until the alcohol evaporates.

In another aspect, the method includes cutting the treated filter paper into sections and affixing at least one section to tissue paper to form a tissue tester sheet.

In another aspect, the method includes contacting the tissue tester with respiratory secretions wherein at least a portion of the tissue tester changes color when illness is indicated.

In another aspect, the color change is from a yellow hue to a brown hue.

In another aspect, the method includes mixing turmeric and alcohol in solution comprises mixing in the ratio of about 20 ml of turmeric to 237 ml of rubbing alcohol.

In another aspect, the method includes soaking filter paper in the solution comprises soaking white filter paper in the solution for up to 30 seconds.

In yet another aspect a method for making an early indication, drug free, self-administered tester to determine when an individual may harbor respiratory pathogens that may induce illness is provided including:

mixing turmeric and alcohol in solution, wherein mixing turmeric and alcohol in solution comprises mixing in the ratio of about 20 ml of turmeric to 237 ml of rubbing alcohol;

soaking filter paper in the solution for up to 30 seconds;

removing the filter paper from the solution;

drying the filter paper until the alcohol evaporates;

cutting the treated filter paper into sections;

affixing at least one section to tissue paper to form a tissue tester sheet; and

contacting the tissue tester with respiratory secretions wherein at least a portion of the tissue tester changes color from a yellow hue to a brown hue when illness is indicated.

### BRIEF DESCRIPTION OF THE DRAWINGS

While the novel features of the invention are set forth with particularity in the appended claims, the invention, both as to organization and content, will be better understood and appreciated, along with other objects and features thereof, from the following detailed description taken in conjunction with the drawings, in which:

FIG. 1 schematically shows one example of tissue tester components.

FIG. 2 schematically shows an alternate example of tissue tester components.

FIG. 3 schematically shows a flow diagram of one example of a method for making a tissue tester.

FIG. 4 schematically shows another example of a tissue tester indicating the presence of illness after exposure to respiratory fluids.

In the drawings, identical reference numbers identify similar elements or components. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

### DETAILED DESCRIPTION OF THE PREFERRED EMBODIMENTS

The following disclosure describes several embodiments for a tissue tester. Several features of methods and systems in accordance with example embodiments are set forth and described in the Figures. It will be appreciated that methods and systems in accordance with other example embodiments can include additional procedures or features different than those shown in the Figures. Example embodiments are described herein with respect to a tissue tester for home use. However, it will be understood that these examples are for the purpose of illustrating the principles, and that the invention is not so limited. Additionally, methods and systems in accordance with several example embodiments may not include all of the features shown in the Figures.

Unless the context requires otherwise, throughout the specification and claims which follow, the word “comprise” and variations thereof, such as, “comprises” and “comprising” are to be construed in an open, inclusive sense that is as “including, but not limited to.”

Reference throughout this specification to “one example” or “an example embodiment,” “one embodiment,” “an embodiment” or combinations and/or variations of these terms means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

### Definitions

Generally, as used herein, the following terms have the following meanings:

As used herein, “plurality” is understood to mean more than one. For example, a plurality refers to at least two, three, four, five, ten, 25, 50, 75, 100, or more.

“Obtaining” is understood herein as manufacturing, purchasing, or otherwise coming into possession of.

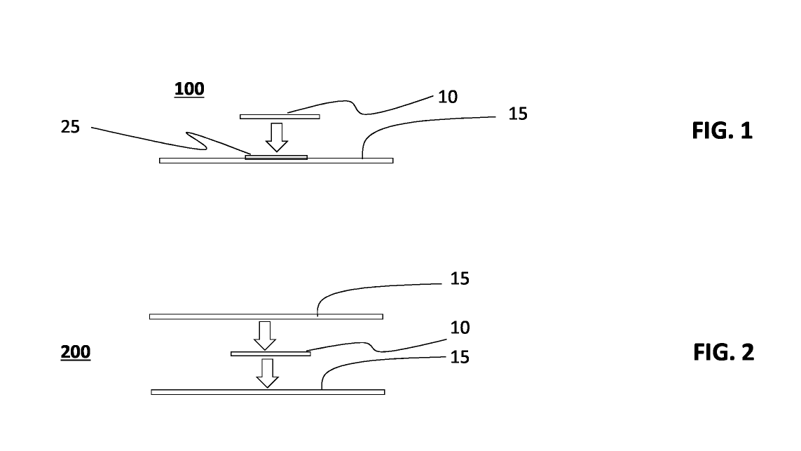

Referring now to FIG. 1 one example of tissue tester components is schematically shown. A treated paper 10 impregnated with a color changing indicator is affixed to a sheet of single ply or multi-ply tissue paper 15 by means of a fixative 25. In one embodiment, the fixative may comprise any convenient adhesive as is well known in the paper making art such as glue, paste, tape or the like. In addition, the treated paper may be affixed by hook and loop material, threading, and/or embossing onto the tissue. When the components are assembled they will appear as a substantially flat, textured sheet of tissue paper to the user.

It will be understood by those skilled in the art having the benefit of this disclosure that the above-described elements may be of any convenient shape, size or configuration and that the invention is not so limited. However, it may be advantageous that the size of the tissue be the standard size of tissue paper or about 20.5 by 18 cm per sheet. The treated paper piece may advantageously be smaller, for example from 2 to 3 times smaller in area. The treated paper may also advantageously be centrally affixed to the tissue paper.

Referring now to FIG. 2, another example of tissue tester components is schematically shown. A treated paper 10 impregnated with a color changing indicator is sandwiched between two opposing sheets of single ply or multi-ply tissue paper 15. The size of the treated paper may be small enough to allow the opposing sheets to bind to each other using processes well known in the art, thereby holding the treated paper 10 in place. Otherwise, fixative may be used to bind the components together as described above.

Those skilled in the art having the benefit of this disclosure will understand how to affix the treated paper section between sheets of multi-ply tissue paper. The manufacture of multi-ply tissues have long been well known in the art, as for example, taught in U.S. Pat. No. 4,469,735, issued Sep. 4, 1984, to Paul D. Trokhan and entitled, “Extensible multi-ply tissue paper product,” which is incorporated by reference. Trokhan teaches an extensible multi-ply tissue paper product having high tensile energy absorption (TEA); high liquid absorbency; and, preferably, high tensile strength efficiency. The product comprises plies which are preferably embossed and discontinuously adhered together.

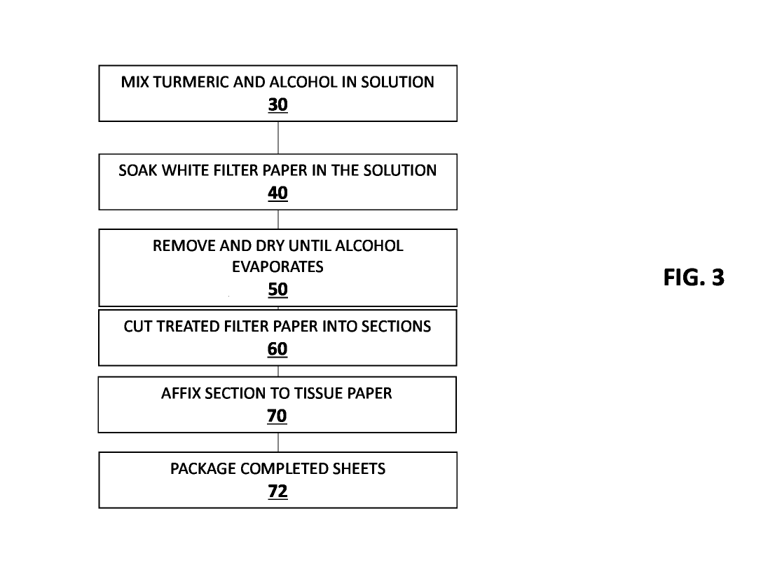

Referring now to FIG. 3, one example of a flow diagram of a method for making a tissue tester is schematically shown. The process is initiated by mixing turmeric and alcohol in solution 30. Then white filter paper is soaked in the solution 40. The white filter paper is removed from the solution and dried until the alcohol evaporates 50. The treated filter paper is cut into sections 60. At least one section of treated paper is affixed to tissue paper 70 to form a tissue tester sheet. Completed sheets of tissue testers may then be packaged for sale 72. In one useful embodiment the treated filter paper may be sized to use as the tester by itself, eliminating the need to cut it into sections and affix it to a sheet of tissue paper used as a carrier.

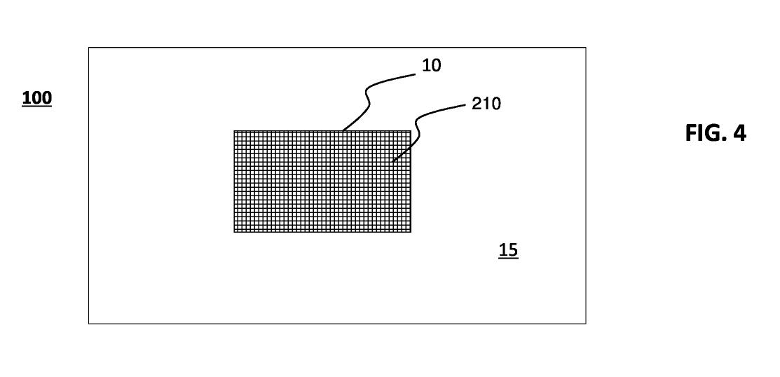

Referring now to FIG. 4, another example of a tissue tester indicating the presence of illness after exposure to respiratory fluids is schematically shown. Here a tissue tester 100 comprises a sheet of tissue paper 15 carrying a section of treated indicator filter paper 10 after exposure to respiratory fluids indicating the presence of illness. The gridded pattern 210 signifies a color change from yellow to brown. Thus observation of the color change to a doctor or lay person may indicate the presence of illness such as a virus or bacterial infection. As indicated above, in an alternate embodiment the tissue paper sheet may be eliminated and the treated filter paper itself can serve as the entire tester.

### WORKING EXAMPLE

As shown below with reference to the example a tester made in accordance with the teachings herein provides a useful early indication of disease for patients as compared with subsequent actual diagnosis and treatment.

In one working example the formulation was as follows:

20 ml (4 teaspoons) of turmeric was mixed with 237 ml (one cup) of rubbing alcohol. White filter paper was then briefly soaked in the solution for up to 30 seconds or until it turned yellow or golden in color. The filter paper may comprise any available generic type filter paper, preferably white, as may be used for coffee filters and the like.

The treated filter paper was air dried for 24 hours to allow the alcohol to evaporate. After drying the filter paper was cut into sizes for affixing onto a generic facial tissue.

A plurality of these samples were made and used experimentally with the results as shown in Table 1. In this working example, the tissue tester was used to collect respiratory secretions from the nose or mouth of each candidate tested. The indicator changed color to a brownish hue in a spot, an area, a plurality of areas or in its entirety when it came in contact with or was exposed to respiratory secretions and pathogens. The pathogens were later diagnosed to be bacterial or viral. Thus the brownish hue indicated the presence of illness.

In the cases where the indicator was exposed to normal respiratory secretions it retained its original yellow or golden color. Thus, the lack of a change to a brownish hue or retention of the yellow or golden color signified non-illness or wellness. In this example 258 subjects were tested. Table 1 lists the results from testing the product made as described above.

### Notes for Table 1:

Some patients appear more than once due to being tested on a return visit.

Patient data for patients 1-27 were discarded to maintain consistency due to a change in testing protocol implemented after patient 27.

Uncertain results are noted by a “?” in the B/V/ø column.

### Legend:

ø=null or negative,

+=positive result or treatment undertaken,

N=Nose sample,

Nø=No nose sample,

M=Mouth sample,

Mø=No mouth sample,

Blank fields indicate no information available for this parameter.

### Headings:

PT#=Patient Number,

Age=Age of patient,

Fever=presence (+) or absence (ø) of fever,

Symt=Symptoms present (+) or absent (ø),

Duration=Duration of symptoms in days (d), weeks (wks.) or months (mos.),

Route=Respiratory sample source, Nose (N) or Mouth (M),

Result=Color change in indicator from original Yellow if any,

B/V/ø=Diagnosis: Bacterial/Viral/none (or as noted),

Treatment=Treatment undertaken (+) or no treatment applied (ø).

TABLE 1

| PT # | Age | Fever | Symt | Duration | Route | Result | B/V/ø | Treatment |
|---|---|---|---|---|---|---|---|---|
| 28 | 35 | ø | + | >14 d | N | Brown | Bacterial | + |
| | | | | | | weakly | | |
| 29 | 39 | ø | + | >10 d | N | Brown | Bacterial | + |
| 30 | 46 | ø | + | >14 d | N | | | |
| | | | | | M | Brown | Bacterial | + |
| 31 | 28 | ø | + | >14 d | | Weakly | Bacterial | + |
| | | | | | | Brown | | |
| 32 | 66 | 99 | + | 3 d | N | Weakly | In | ø |
| | | | | | | Brown | progress? | |
| | | | | | M | | | * |
| 33 | 57 | 99 | + | >28 d | N | Brown | Bacterial | + |
| 34 | 69 | 100 | + | >14 d | N | Brown | Bacterial | + |
| 35 | 43 | ø | ø | ø | N | Yellow | ø | ø |
| | | | | | M | Yellow | | |
| 36 | 39 | ø | ø | ø | N | Yellow | ø | ø |
| | | | | | M | Yellow | | |
| 37 | 61 | 99 | + | 2 d | N | Weakly | Viral? | |
| | | | | | | Brown | | |
| | | | | | M | | ø | ø |
| 38 | 51 | + | + | >10 d | N | Brown | Bacterial | + |
| 39 | 51 | ø | + | >7 d | N | Brown | Bacterial | + |
| 40 | 18 | 99.6 | + | | N | Brown | Bacterial | + |
| 41 | 54 | ø | + | 2 d | N | Brown | Viral | ø |
| | | | | | | early? | | |
| 42 | 70 | ø | + | 7 d | N | Brown | | |
| | | | | | M | Brown | Bacterial | + |
| 43 | 64 | + | + | 5 d | N | Brown | | |
| | | | | | M | Brown | Bacterial | + |
| 44 | 53 | ø | + | 28 d | N | Yellow | ø | ø |
| | | | | | M | Yellow | Residual | * |
| | | | | | | | Viral? | |
| 45 | 52 | ø | + | 5 d | N | Yellow | | |
| | | | | | M | Yellow | Viral | ø |
| 46 | 64 | ø | + | 21 d | N | Yellow | | |
| | | | | | M | Yellow | Viral | ø |
| 47 | 36 | ø | + | >14 d | N | Brown | | |
| | | | | | M | Brown | Bacterial | + |
| 48 | 38 | ø | + | 14 d | N | Yellow | | |
| | | | | | M | Yellow | Viral | ø |
| 49 | 59 | 99 | + | 3 d | N | Yellow | | |
| | | | | | M | Yellow | Viral | ø |
| 50 | 78 | ø | + | 21 d | N | Yellow | | |
| | | | | | M | Yellow | Viral? | ø |
| 51 | 51 | ø | + | 2 d | N | Yellow | | |
| | | | | | M | Brown? | Viral? | ø |
| 52 | 37 | ø | + | 7 d | N | Yellow | | |
| | | | | | M | Yellow | Viral | ø |
| 53 | 50 | ø | | 7 d | N | Brown | | |
| | | | | | M | Yellow | Bacterial | + |
| 54 | 73 | ø | ø | ø | N | Yellow | ø | ø |
| | | | | | M | Yellow | | |
| 55 | 44 | ø | + | 21 d | N | Yellow | ø | |
| | | | | | M | Yellow | Allergies | φ |
| 56 | 65 | ø | Vague | 28 d | N | Yellow | | |
| | | | | | M | Yellow | ø | ø |
| 57 | 15 | ø | ø | ø | N | Yellow | ø | ø |
| 58 | 51 | 99.8 | + | >4 d | N | Brown | Bacterial | + |
| | | | | | M | | | |
| 59 | 15 | 991 | + | >3 d | N | Brown | Bacterial | + |
| | | | | | M | | | |
| 60 | 49 | ø | + | >14 d | N | Slightly | | |
| | | | | | | Brown | | |
| | | | | | M | Brown | Bacterial | + |
| 61 | 55 | ø | + | 3 d | N | Brown | | |
| | | | | | M | Yellow | Bacterial | + |
| 62 | 46 | ø | + | 11 d | N | Yellow | Residual | |
| | | | | | | | ** | |
| | | | | | M | Slightly | Bacterial | + |
| | | | | | | Brown | | |
| | | | | | | Spot | | |
| 63 | 58 | ø | + | 14 d | N | Brown | Bacterial | + |
| | | | | | M | | | |
| 64 | 27 | ø | + | 8 d | N | Brown | Bacterial | + |
| | | | | | | spots | | |
| | | | | | Mø | | | |
| 65 | 51 | ø | + | 10 d | N | Yellow | | |
| | | | | | M | Yellow | Viral? | ø |
| 66 | 27 | 99.1 | + | 10 d | N | Brown | | |
| | | | | | M | Yellow | Bacterial | + |
| 67 | 44 | ø | + | >8 wks. | N | Brown | Bacterial | + |
| | | | | | Mø | | | |
| 68 | 52 | ø | + | 2½-3 wks. | N | Brown | Bacterial | + |
| | | | | | Mø | | | |
| 69 | 31 | ø | + | 5 d | N | Brown | Bacterial | + |
| | | | | | Mø | | | |
| 70 | 22 | ø | + | 2-3 Mos. | N | Brown | Bacterial | + |
| | | | | | Mø | | | |
| 71 | 31 | ø | + | 2-3 wks. | N | Brown | Bacterial | + |
| | | | | | M | Yellow | | |
| 72 | 70 | 99.1 | + | 28 d | N | Brown | Bacterial | + |
| | | | | | M | Yellow | | |
| 73 | 64 | ø | + | 7 d | N | Brown | Bacterial | + |
| | | | | | M | Brown | | |
| 74 | 65 | ø | + | 10 d | N | Yellow | ø | ø |
| | | | | | | | Asthma/ | |
| | | | | | | | Viral | |
| | | | | | M | Yellow | | |
| 75 | 57 | 99.3 | + | 7 d | N | Yellow | Bacterial | + |
| | | | | | M | Brown | | |
| 76 | 31 | ø | ± | 3 d | N | Brown | Early | + |
| | | | | | | | Bacterial | |
| | | | | | M | Brown | | |
| 77 | 53 | ø | ø | ø | N | Yellow | ø | ø |
| | | | | | M | Yellow | | |
| 78 | 53 | ø | + | 2 wks. | N | Yellow | Viral? | ø |
| | | | | | M | Yellow | | |
| 79 | 52 | ø | + | >2 wks. | N | Brown | Bacterial | + |
| | | | | | Mø | | | |
| 80 | 54 | ø | + | Months | N | Few | ? | ø |
| | | | | | | specks | | |
| | | | Chronic | | Mø | Yellow | | |
| 81 | 55 | ø | + | Weeks | N | Specks | ø | ø |
| | | | | | Mø | | | |
| 82 | 40 | 99.5 | ø | ø | N | spots--1 | Early | ø |
| | | | | | | brown | Bacterial | |
| | | | | | Mø | | | |
| 83 | 46 | 99.6 | + | 1 d | N | Brown | Early? | ø |
| | | | | | | spots | | |
| | | | | | Mø | | | |
| 84 | 54 | 101.4 | + | 5 d | N | Brown | Bacterial | + |
| | | | | | M | Brown | | |
| 85 | 53 | ø | + | >1 yr. | N | Brown | | |
| | | | | | | spots | | |
| | | | | | M | Brown! | Bacterial | + |
| 86 | 32 | 99.6 | + | 3 d | N | Brown | Bacterial | + |
| | | | | | M | Brown | | |
| 87 | 42 | ø | + | >4 wks. | N | Brown | Bacterial | + |
| | | | | | M | Mild | | |
| 88 | 31 | 99 | + | 1.5 wks. | N | Brown | Bacterial | + |
| | | | | | Mø | | | |
| 89 | 43 | ø | + | weeks | N | Brown | Early | ø |
| | | | | | | spots | Bacterial | |
| | | | | | M | Yellow | | |
| 90 | 87 | ø | + | 4 wks. | N | Yellow | Viral? | ø |
| | | | | | Mø | | | |
| 91 | 63 | ø | + | >2 wks. | N | Yellow | Bacterial | + |
| | | | | | M | Brown | Bacterial | |
| 92 | 61 | ø | + | 5 d | N | Brown | Bacterial | + |
| | | | | | M | Brown | | |
| 93 | 51 | ø | + | 5 d | N | Brown | Bacterial | + |
| | | | | | Mø | | | + |
| 94 | 64 | ø | + | Tx'd | N | Brown | Residual | + |
| | | | | Mar. 4, 2010 | | spots | | |
| | | | | | Mø | | Bacterial | |
| 95 | 31 | ø | + | 3 d | N | Brown | Bacterial | + |
| | | | | | Mø | | | |
| 96 | 51 | ø | + | >3 wks. | N | Brown | Bacterial | + |
| | | | | | | spots | | |
| | | | | | Mø | | | |
| 97 | 65 | ø | ø | ø | N | Yellow | ø | ø |
| | | | | | Mø | | | |
| 98 | 78 | ø | + | >4 wks. | Nø | | | |
| | | | | | M | Brown | Bacterial | + |
| 99 | 45 | ø | ø | 3 d | N | Brown | ø | ø |
| | | | | | | spot only | | |
| | | | | | Mø | | | |
| 100 | 30 | ø | + | 2 wks. | N | Brown | ø | ø |
| | | | | | | spot only | | |
| | | | | | M | Yellow | | |
| 101 | 53 | ø | + | >2 wks. | N | Brown | Bacterial | + |
| | | | | | Mø | | | |
| 102 | 58 | ø | ø | ø | N | Yellow | ø | ø |
| | | | | | Mø | | | |
| 103 | ? | ø | + | >4 wks. | N | Brown | Bacterial | + |
| | | | | | Mø | | | |
| 104 | 46 | ø | ø | ø | N | Yellow | ø | Patient |
| | | | | | | | | treated |
| | | | | | | | | later |
| | | | | | Mø | | | ø |
| 105 | 27 | ø | + | >4 wks. | N | Brown | Bacterial | + |
| | | | | | M | Yellow | | |
| 106 | 62 | ø | ø | ø | N | 1 spot | ø | ø |
| | | | | | | only | | |
| | | | | | Mø | | | |
| 107 | 42 | 99 | + | 2 d | N | Brown | Bacterial | + |
| | | | | | M | Brown | | |
| 108 | 53 | ø | ø | ø | N | Spot | ø | ø |
| | | | | | | only | | |
| | | | | | M | Yellow | | |
| 109 | 31 | ø | ø | ø | N | Spot | ø | ø |
| | | | | | M | Yellow | | |
| 110 | 46 | ø | ø | ø | N | Yellow | ø | ø |
| | | | | | M | Yellow | | |
| 111 | 42 | ø | ø | ø | N | Spot | ø | ø |
| | | | | | M | Yellow | | |
| 112 | 54 | ø | ø | ø | N | Yellow | ø | ø |
| | | | | | M | Yellow | | |
| 113 | 41 | ø | + | >weeks | N | Yellow | ø | ø |
| | | | | | M | Yellow | | |
| 114 | 20 | ø | ø | ø | N | Spot | ø | ø |
| | | | | | M | Yellow | | |
| 115 | 44 | ø | + | >4 wks. | N | Brown | Bacterial | + |
| | | | | | M | Yellow | | |
| 116 | 54 | ø | ø | ø | N | Yellow | ø | ø |
| | | | | | M | Yellow | | |
| 117 | 41 | ø | + | >11 d | N | Brown | Bacterial | + |
| | | | | | M | Yellow | | |
| 118 | 70 | 99 | + | >weeks | N | Brown | Bacterial | + |
| | | | | | M | Yellow | | |
| 119 | 52 | ø | ø | ø | N | Yellow | Early | ø |
| | | | | | | | Bacterial? | |
| | | | | | M | Brown | | |
| 120 | 42 | ø | + | 2 wks. | N | Brown | Bacterial | + |
| | | | | | M | Brown | | |
| 121 | 58 | ø | + | wks. | N | Yellow | Viral | ø |
| | | | | | M | Yellow | | |
| 122 | 20 | 99.2 | + | 4 d | N | Spot | Viral? | ø |
| | | | | | | only | | |
| | | | | | M | Yellow | Early | |
| | | | | | | | Bacterial? | |
| 123 | 61 | ø | + | 2 d | N | Spot | Viral? | ø |
| | | | | | | only | | |
| | | | | | M | Yellow | | |
| 124 | 46 | 99.9 | + | 2 d | N | Yellow | Viral | ø |
| | | | | | Mø | | | |
| 125 | 54 | 99 | + | 2 d | N | Spot | Viral? | ø |
| | | | | | | only | | |
| | | | | | M | Yellow | Early | |
| | | | | | | | Bacterial? | |
| 126 | 51 | ø | ø | ø | N | Yellow | ø | ø |
| | | | | | M | Yellow | | |
| 127 | 43 | ø | + | >2 wks. | N | Brown | Bacterial | + |
| | | | | | M | Yellow | | |
| 128 | 58 | ø | + | >2 wks. | N | Brown | Bacterial | + |
| | | | | | M | Yellow | | |
| 129 | 53 | 99.2 | + | 2 d | N | Yellow | Viral | ø |
| | | | | | Mø | | | |
| 130 | 26 | 100.2 | + | 4 d | N | Brown | Bacterial | + |
| | | | | | M | Brown | | |
| 131 | 18 | ø | + | >7 d | N | Brown | Bacterial | + |
| | | | | | M | Yellow | | |
| 132 | ? | ø | ± | 5 d | N | Brown | Viral? | ø |
| | | | | | | spots | | |
| | | | | | Mø | | Early | |
| | | | | | | | Bacterial? | |
| 133 | 54 | 100.9 | + | >3 d | N | Brown | Bacterial | + |
| | | | | | M | Brown | | |
| 134 | 51 | ø | ø | ø | N | Yellow | ø | ø |
| | | | | | M | Yellow | | |
| 135 | 18 | ø | ø | ø | N | Yellow | ø | ø |
| | | | | | Mø | | | |
| 136 | 14 | ø | ø | ø | Nø | | | |
| | | | | | M | Yellow | ø | ø |
| 137 | ? | ø | + | 2 d | N | Brown | Bacterial | + |
| | | | | | Mø | | | |
| 138 | 58 | 99.2 | + | 2 d | N | Yellow | Viral | ø |
| | | | | | Mø | | | |
| 139 | 35 | ø | ± | Few days | N | Brown | Getting Bacterial | ø |
| | | | | | Mø | | | |
| 140 | 28 | ø | ø | ø | N | Brown | ø | ø |
| | | | | | Mø | | | |
| 141 | 35 | ø | + | >4 wks. | N | Brown | Bacterial | + |
| | | | | | Mø | | | |
| 142 | 70 | ø | + | >few wks. | N | Brown | Bacterial | + |
| | | | | | M | Brown | | |
| 143 | 24 | ø | + | >4 wks. | N | Lots of | Bacterial | + |
| | | | | | | brown | | |
| | | | | | | spots | | |
| | | | | | Mø | | | |
| 144 | 46 | ø | + | >4 d | N | Brown | Bacterial | + |
| | | | | | M | Yellow | | |
| 145 | 56 | 99.9 | + | 2 d | N | Yellow | Viral | ø |
| | | | | | M | Yellow | | |
| 146 | 59 | 99 | + | wks. | N | Yellow | Viral | ø |
| | | | | | M | Yellow | | |
| 147 | 21 | ø | + | 4 d | N | Brown | Bacterial | + |
| | | | | | M | Brown | | |
| 148 | 44 | 99.2 | + | wks. | N | Spot | Viral | ø |
| | | | | | Mø | | | |
| 149 | 54 | 99 | + | 1 wk. | N | Brown | Bacterial | + |
| | | | | | M | Brown | | |
| 150 | 54 | ø | ø | ø | N | 1 spot | ø | ø |
| | | | | | | only | | |
| | | | | | M | Yellow | | |
| 151 | 56 | ø | + | 6 wks. | N | Yellow | ø | ø |
| | | | | | M | Yellow | | |
| 152 | 63 | 99.1 | + | few wks. | N | Spot | Bacterial | Pt declined |
| | | | | | | brown | | |
| | | | | | M | Brown | | |
| 153 | 20 | 99.1 | + | few wks. | N | Spot | Bacterial | + |
| | | | | | | brown | | |
| | | | | | M | Yellow | | |
| 154 | 54 | — | ø | ø | Nø | | | |
| | | | | | M | Yellow | ø | ø |
| 155 | 39 | ø | + | 5 d | N | Brown | Bacterial | + |
| | | | | | | spots | | |
| | | | | | M | Yellow | | |
| 156 | 49 | 100.9 | + | >5 d | N | Brown | Bacterial | + |
| | | | | | Mø | | | |
| 157 | 32 | 99.4 | ø | ø | N | Yellow | ø | ø |
| | | | | | Mø | | | |
| 158 | 22 | ø | + | >2 d | Nø | | Probable | + |
| | | | | | | | Bacterial | |
| | | | | | M | Yellow | Symptoms? | |
| 159 | 79 | ø | + | 2 wks. | N | Spots | Viral | ø |
| | | | | | Mø | | | |
| 160 | 28 | ø | + | >2 wks. | N | Brown | Bacterial | + |
| | | | | | | spots | | |
| | | | | | M | Yellow | Sinus | |
| 161 | 23 | ø | + | 1 wk | N | Yellow | Viral | ø |
| | | | | | M | Yellow | | |
| 162 | 29 | ø | + | 1 wk | N | Brown | Bacterial | + |
| | | | | | M | Brown | | |
| 163 | 25 | 99.1 | ø | ø | Nø | | | |
| | | | | | M | Yellow | Viral | ø |
| 164 | 79 | ø | + | 4 wks. | N | Yellow | Viral | ø |
| | | | | | M | Yellow | | |
| 165 | 64 | ø | + | 3 wks. | N | Brown | Bacterial | + |
| | | | | | M | Brown | | |
| 166 | ? | 99 | + | Few | N | Yellow | Viral | ø |
| | | | | days | | | | |
| | | | | | M | Yellow | | |
| 167 | 30 | ø | ø | ø | N | Yellow | ø | ø |
| | | | | | M | Yellow | | |
| 168 | 32 | ø | + | wks. | N | Slightly | Bacterial | + |
| | | | | | | brown | | |
| | | | | | M | Slightly | | |
| | | | | | | brown | | |
| 169 | 52 | ø | + | 7 d | Nø | | | |
| | | | | | M | Yellow | Viral | ø |
| 170 | ? | ø | ø | ø | N | 1 spot | Viral? | ø |
| | | | | | | only | | |
| | | | | | M | Yellow | | |
| 171 | 11 | ø | ø | ø | N | Yellow | ø | ø |
| | | | | | M | Yellow | | |
| 172 | 64 | ø | ø | ø | N | Yellow | | |
| | | | | | M | Yellow | ø | ø |
| 173 | 47 | ø | + | 3-4 wks. | Nø | | | |
| | | | | | M | Brown | Bacterial | + |
| 174 | 51 | 99 | + | 8 wks. | N | Yellow | | |
| | | | | | M | Brown | Bacterial | + |
| 175 | 57 | 99 | + | >8 wks. | N | Spot | Bacterial | + |
| | | | | | | brown | | |
| | | | | | M | Brown | | |
| 176 | 60 | ø | + | 4 wks. | N | Yellow | Viral/ø | ø |
| | | | | | M | Yellow | | |
| 177 | 65 | ø | ø | ø | N | Spot | ø | ø |
| | | | | | Mø | | | |
| 178 | 56 | ø | ø | ø | N | Yellow | | |
| | | | | | Mø | | ø | ø |
| 179 | 59 | 99.2 | ø | ø | N | Spot | Viral? | ø |
| | | | | | Mø | | | |
| 180 | 53 | ø | ø | ø | N | Yellow | ø | ø |
| | | | | | M | Yellow | | |
| 181 | 57 | ø | ø | ø | N | 1 spot | ø | ø |
| | | | | | | only | | |
| | | | | | Mø | | | |
| 182 | 47 | 99.3 | + | >4 wks. | N | Yellow | Bacterial | + |
| | | | | | M | 5 light | | |
| | | | | | | brown | | |
| 183 | 64 | ø | ø | ø | N | Yellow | ø | ø |
| | | | | | M | Yellow | | |
| 184 | 55 | 99.9 | ø | ø | N | Yellow | Viral? | ø |
| | | | | | M | Yellow | | |
| 185 | 21 | ø | + | 7 d | N | Spots | Viral | ø |
| | | | | | M | Yellow | | |
| 186 | 29 | 99 | + | 3 d | N | spots | Viral? | ø |
| | | | | | M | Yellow | | |
| 187 | 54 | ø | ø | ø | N | Very | Early | ø |
| | | | | | | slight | Bacterial? | |
| | | | | | | Brown | | |
| | | | | | M | Very | | |
| | | | | | | slight | | |
| | | | | | | Brown | | |
| 188 | 35 | ø | ø | ø | N | Yellow | ø | ø |
| | | | | | Mø | | | |
| 189 | 29 | 99 | + | wks. | N | Yellow | Viral? | ø |
| | | | | | Mø | | | |
| 190 | 55 | 99 | ø | ø | N | Yellow | Viral? | ø |
| | | | | | M | Yellow | | |
| 191 | 30 | ø | + | 2 d | N | Slightly | Early | ø |
| | | | | | | brown | Bacterial? | |
| | | | | | M | Slightly | Viral? | |
| | | | | | | brown | | |
| 192 | 32 | 99 | ø | ø | N | Yellow | Viral? | ø |
| | | | | | Mø | | | |
| 193 | 73 | ø | + | 1 wk. | N | Spot | Viral? | ø |
| | | | | | | only | | |
| | | | | | M | Yellow | | |
| 194 | 73 | ø | ø | ø | N | Yellow | ø | ø |
| | | | | | M | Yellow | | |
| 195 | 29 | 99 | + | 1 wk. | N | Yellow | Viral? | ø |
| | | | | | M | Yellow | | |
| 196 | 18 | 99.2 | + | 8 d | N | Spot | Bacterial | + |
| | | | | | | Brown | | |
| | | | | | M | Yellow | | |
| 197 | 27 | ø | ø | ø | N | Yellow | ø | ø |
| | | | | | M | Yellow | | |
| 198 | 46 | 99.4 | + | 7 d | N | Brown | Bacterial | + |
| | | | | | M | Brown | | |
| 199 | 74 | ø | ø | ø | N | Yellow | ø | ø |
| | | | | | M | Yellow | | |
| 200 | 65 | 99 | ø | ø | N | Yellow | ø | ø |
| | | | | | M | Yellow | | |
| 201 | 31 | 99.1 | + | 5 d | N | Brown | Bacterial | + |
| | | | | | M | Brown | | |
| 202 | 63 | ø | + | 7 d | N | Spot | Bacterial | + |
| | | | | | | brown | | |
| | | | | | M | Brown | | |
| 203 | 21 | 100.5 | + | 4 wks. | Nø | | | |
| | | | | | M | Brown | Bacterial | + |
| 204 | 37 | ø | + | >4 Mos. | N | Brown | Bacterial | + |
| | | | | | M | Brown | | |
| 205 | 31 | 99 | + | 2 d | Nø | | | |
| | | | | | M | Yellow | ø | ø |
| 206 | 25 | ø | + | 7 d | N | Brown | Bacterial | + |
| | | | | | | spots | | |
| | | | | | M | Yellow | | |
| 207 | 18 | 99.5 | + | 2 wks. | N | Spot | Bacterial | + |
| | | | | | | brown | | |
| | | | | | M | Yellow | | |
| 208 | 21 | ø | ø | ø | N | Spot | ø | ø |
| | | | | | | Brown | | |
| | | | | | M | Yellow | | |
| 209 | 51 | ø | + | 2 mos. | N | Brown | Getting | ø |
| | | | | | | | sick? | |
| | | | | | M | Brown | | |
| 210 | 32 | ø | + | 3 d | N | Brown | Bacterial | + |
| | | | | | Mø | | | |
| 211 | 31 | ø | + | 10 d | N | Brown | Bacterial | + |
| | | | | | M | Brown | | |
| 212 | 38 | ø | + | 14 d | N | Brown | Bacterial | + |
| | | | | | M | Brown | | |
| 213 | 51 | ø | ø | ø | N | Yellow | ø | ø |
| | | | | | M | Yellow | | |
| 214 | 50 | ø | + | 7 d | N | Brown | Bacterial | + |
| | | | | | M | Brown | | |
| 215 | 27 | ø | + | 4 d | N | Yellow | ø | ø |
| | | | | | Mø | | | |
| 216 | 74 | ø | + | 6 d | N | Brown | Bacterial | + |
| | | | | | | spots | | |
| | | | | | M | Yellow | | |
| 217 | 49 | 99.3 | ø | ø | N | Yellow | Viral? | ø |
| | | | | | M | Yellow | | |
| 218 | 28 | 99.3 | + | 7 d | N | Spots | Bacterial | + |
| | | | | | M | Brown | | |
| 219 | 52 | 100.2 | + | 5 d | N | Brown | Bacterial | + |
| | | | | | | spots | | |
| | | | | | M | Yellow | | |
| 220 | 41 | ø | + | 5 wks. | N | Brown | Bacterial | + |
| | | | | | M | Yellow | | |
| 221 | 54 | ø | + | >6 d | N | Brown | Bacterial | + |
| | | | | | M | Yellow | | |
| 222 | 50 | ø | + | >2 wks. | N | Brown | Bacterial | + |
| | | | | | M | Spots | | |
| 223 | 57 | 99.1 | + | >1 wk | N | Brown | Bacterial | + |
| | | | | | M | Slight | | |
| 224 | 56 | ø | + | 7 d | N | Yellow | Viral | ø |
| | | | | | M | Yellow | | |
| 225 | 30 | ø | + | >4 wks. | N | Brown | Bacterial | + |
| | | | | | M | Brown | | |
| 226 | 47 | ø | + | 7 d | N | Brown | Bacterial | + |
| | | | | | M | Brown | | |
| 227 | 44 | ø | + | 5 d | N | Brown | Bacterial | + |
| | | | | | M | Brown | | |
| 228 | 51 | ø | + | >4 wks. | N | Spots | Bacterial | + |
| | | | | | M | Brown | | |
| 229 | 30 | ø | + | 5 d | N | Spots | Viral? | ø |
| | | | | | | only | | |
| | | | | | M | Yellow | Early | |
| | | | | | | | Bacterial? | |
| 230 | 30 | ø | + | 6 d | N | Yellow | Viral | ø |
| | | | | | M | Yellow | | |
| 231 | 70 | ø | + | >4 wks. | N | Brown | Bacterial | + |
| | | | | | M | Brown | | |
| 232 | 15 | ø | + | >2 wks. | N | Brown | Bacterial | + |
| | | | | | | spots | | |
| | | | | | M | Yellow | | |
| 233 | 49 | 99.3 | + | 7 d | N | Spots | | |
| | | | | | M | Brown | Bacterial | + |
| 234 | 15 | ø | + | >3 wks. | N | Spots | | |
| | | | | | M | Brown | Bacterial | + |
| 235 | 61 | ø | + | >7 d | N | Spots | Bacterial | + |
| | | | | | Mø | | | |
| 236 | 57 | ø | + | 4 d | N | Yellow | Viral | ø |
| | | | | | M | Yellow | | |
| 237 | 44 | ø | + | 7 d | N | Spots | Bacterial | + |
| | | | | | M | Yellow | | |
| 238 | 18 | 99.3 | ø | ø | N | Yellow | Viral | ø |
| | | | | | M | Yellow | | |
| 239 | 56 | ø | ø | ø | N | 1 spot | ø | ø |
| | | | | | | only | | |
| | | | | | M | Yellow | | |
| 240 | 82 | ø | ø | ø | N | 1 spot | ø | ø |
| | | | | | | only | | |
| | | | | | M | Yellow | | |
| 241 | 54 | ø | ø | ø | N | Yellow | ø | ø |
| | | | | | M | Yellow | | |
| 242 | 26 | 99.4 | + | 5 d | N | Brown | Bacterial | + |
| | | | | | | streaks | | |
| | | | | | M | Yellow | | |
| 243 | 58 | 99.4 | + | 5 d | N | Spots | Bacterial | + |
| | | | | | M | Brown | | |
| 244 | 51 | 99.2 | + | 4 wks. | N | Brown | Bacterial | + |
| | | | | | M | Yellow | | |
| 245 | 51 | ø | + | 3 wks. | N | Spot | Early | |
| | | | | | | only | Bacterial? | ø |
| | | | | | M | Yellow | Allergies? | |
| 246 | 18 | ø | ø | ø | N | Yellow | ø | ø |
| | | | | | M | Yellow | | |
| 247 | 61 | 99 | + | 7 d | N | Yellow | Allergies? | ø |
| | | | | | M | Yellow | Viral? | |
| 248 | 57 | 99.2 | + | wks. | N | Yellow | | |
| | | | | | M | Brown | Bacterial | + |
| 249 | 10 | 99.2 | + | 2 d | N | Yellow | Allergies? | ø |
| | | | | | M | Yellow | Viral? | |
| 250 | 41 | ø | + | 2 wks. | N | Yellow | Viral? | ø |
| | | | | | M | Yellow | Allergies? | |
| 251 | 64 | ø | + | Few | N | Yellow | Allergies? | ø |
| | | | | mos. | | | | |
| | | | | | M | Yellow | | |
| 252 | 61 | 99.7 | + | 2 wks. | N | Brown | | |
| | | | | | M | Slight | Bacterial | + |
| | | | | | | Brown | | |
| 253 | 46 | 99.5 | + | 2 wks. | N | Yellow | Viral | ø |
| | | | | | M | Yellow | | |
| 254 | 19 | 99.4 | + | 1 wk. | N | Brown | | |
| | | | | | M | Yellow | Bacterial | + |
| 255 | 52 | 98.7 | + | 1 wk. | N | Brown | Bacterial | + |
| | | | | | M | Yellow | | |
| 256 | 26 | 99.6 | + | 6 d | N | Brown | Bacterial | + |
| | | | | | M | Yellow | | |
| 257 | 21 | 99 | + | 4 wks. | N | Brown | Bacterial | + |
| | | | | | M | Yellow | | |
| 258 | 69 | ø | + | 3 d | N | Brown | Bacterial | + |
| | | | | | M | Yellow | | |

The invention has been described herein in considerable detail in order to comply with the Patent Statutes and to provide those skilled in the art with the information needed to apply the novel principles of the present invention, and to construct and use such exemplary and specialized components as are required. However, it is to be understood that the invention may be carried out by different equipment, and devices, and that various modifications, both as to the equipment details and operating procedures, may be accomplished without departing from the true spirit and scope of the present invention.

What is claimed is:

1. A tissue tester for providing an indication of the presence of disease comprising:
a first paper tissue sheet; and
a filter patch treated with a color changing indicator affixed to the first paper tissue sheet.

2. The tissue tester of claim 1, wherein the filter patch is juxtaposed between the first paper tissue sheet and a second paper tissue sheet to form a tissue tester.

3. The tissue tester of claim 1 wherein the treated filter patch is treated in a solution of turmeric and alcohol.

4. The tissue tester of claim 1 wherein the treated filter patch is affixed to a sheet of single ply or multi-ply tissue paper by a fixative selected from the group consisting of adhesive, glue, paste, tape, threading, by hook and loop material, threading, embossing and combinations thereof.

5. The tissue tester of claim 1 wherein the treated filter patch comprises an area smaller than the first paper tissue sheet.

6. The tissue tester of claim 1 wherein the treated filter patch is treated in a solution having a ratio of about 20 ml turmeric to about 237 ml alcohol.

7. The tissue tester of claim 1 wherein the treated filter patch comprises white filter paper.

8. A method for making an early indication, drug free, self-administered tester patch for use in a tester to determine when an individual may harbor respiratory pathogens that may induce illness comprising:
mixing turmeric and alcohol in solution;
soaking filter paper in the solution;
removing the filter paper from the solution; and
drying the filter paper until the alcohol evaporates.

9. The method of claim 8 further comprising cutting the treated filter paper into sections and affixing at least one section to tissue paper to form a tissue tester sheet.

10. The method of claim 9 further comprising contacting the tissue tester with respiratory secretions wherein at least a portion of the tissue tester changes color when illness is indicated.

11. The method of claim 10 wherein the color change is from a yellow hue to a brown hue.

12. The method of claim 8 wherein mixing turmeric and alcohol in solution comprises mixing in the ratio of about 20 ml of turmeric to 237 ml of rubbing alcohol.

13. The method of claim 8 wherein soaking filter paper in the solution comprises soaking white filter paper in the solution for up to 30 seconds.

14. A method for making a self-administered tester to determine when an individual may harbor respiratory pathogens that may induce illness comprising:
mixing turmeric and alcohol in solution, wherein mixing turmeric and alcohol in solution comprises mixing in the ratio of about 20 ml of turmeric to 237 ml of rubbing alcohol;
soaking filter paper in the solution for up to 30 seconds;
removing the filter paper from the solution;
drying the filter paper until the alcohol evaporates;
cutting the treated filter paper into sections;
affixing at least one section to tissue paper to form a tissue tester sheet; and
contacting the tissue tester with respiratory secretions wherein at least a portion of the tissue tester changes color from a yellow hue to a brown hue when illness is indicated.

15. A tissue tester product made by the process of claim 14.

16. A tester for providing an indication of the presence of disease comprising a filter paper sheet treated in a solution of turmeric and alcohol having a ratio of about 20 ml turmeric to about 237 ml alcohol.

17. The tester of claim 16 wherein the treated filter paper comprises white filter paper.

18. The tester of claim 16 wherein at least a portion of the filter paper sheet changes color when exposed to respiratory excretions secretions containing a pathogen.

19. The tester of claim 18 wherein the color change is from a yellow hue to a brown hue.

20. The method of claim 16 wherein the filter paper sheet is treated by soaking it in the solution for up to 30 seconds.
